(19) United States
(12) Patent Application Publication
(10) Pub. No.: US 2006/0222719 A1
(43) Pub. Date: Oct. 5, 2006

(54) Articles of manufacture made from agave residue, and methods for making such articles

(76) Inventors: Eaman Ochun Tang, Rowland Heights, CA (US); David Chi-Ping Chow, Walnut Creek, CA (US)

Correspondence Address: GARY C. HONEYCUTT, 4728 RAVENDALE, RICHARDSON, TX, 75082, US

(21) Appl. No.: 11/098,299

(22) Filed: Apr. 4, 2005

(51) Int. Cl. A61K 36/00

(52) U.S. Cl. 424/725

(57) ABSTRACT

Articles of manufacture are produced from a composition comprising agave plant residue and a thermosetting polymer resin.

### FIELD OF THE INVENTION

The present invention relates to thermosetting polymer resin compositions and to molded articles made from a mixture of such compositions with other materials.

### BACKGROUND OF THE INVENTION

The curing of thermosetting resin compositions results in irreversible chemical changes, transforming a fusible and soluble material into a composite that is infusible and insoluble through the formation of covalently cross-linked, thermally stable, three-dimensional networks, generally known as “plastics.” Such plastics are widely used to make heat-resistant articles that remain stable at 500 degrees F., for example. Common types include polyurethane, phenols, melamine-formaldehyde, urea-formaldehyde, and amino resins. The addition of various fillers and other additives to make reinforced plastics is also well known.

### SUMMARY OF THE INVENTION

Tequila is a well-known alcoholic drink product obtained by fermentation of the blue agave plant, which contains large organic molecules and long fibers. After blue agave is processed to produce tequila, there remains a large amount of fibrous residue from the blue agave plant. Usually, the residue is discarded as waste. However, in accordance with the present invention, we have found that the residue is very useful to produce a variety of new products. In addition to the species known as “blue” agave, the other species of the agave plant yield a similar residue, which is also used in accordance with this invention.

Mixing and reacting agave residue with a thermosetting polymer resin creates many new products having a variety of useful properties. These new products are structurally very hard, durable, insoluble, heat-resistant, and shatter-proof, due to the fiber reinforcement. They are also biodegradable, due to the biodegradable nature of the agave residue.

One embodiment of the invention is a composition of matter comprising agave residue and a thermosetting polymer.

When cured in a mold, such compositions form articles of manufacture, which is another embodiment of the invention. Such articles include home furnishing products, and kitchen-wares, including plates, cups, saucers, bowls, and other dishes, vases, and storage containers. Commercial and industrial articles are also included, such as building blocks, roof tile, ceiling and wall panel, floor tile, cabinet door, window and door frames, counter tops for structural and decorative uses.

Another aspect of the invention includes methods for making such articles of manufacture. For example, one method comprises the steps of grinding the agave residue to a particle size of about 40 to 100 microns in diameter, followed by mixing about 3 parts by weight of the ground agave fiber with about 2 parts by weight of an melamine-formaldehyde resin, and about 1 part by weight calcium carbonate. The mixture is thoroughly blended and then added to a mold that is preheated to about 90 to 130 degrees C. After a cure time of about 2 to 7 minutes, the article is removed from the mold, and may be polished to provide a smooth, glossy finish.

Other fillers and additives may be included in the compositions and articles of the invention. The nature of agave residue is identified and discussed below, along with the preferred resins, fillers and additives.

Agave Residue

Tequila is North America's first alcoholic drink During their exploration of the New World in the late 1400's and early 1500's, Spanish conquistadors discovered a fermented beverage called “pulque” that was produced by the Nahuatl. The Nahuatl are the original inhabitants of the area of western Mexico who primarily used pulque in religious ceremonies and for medicinal purposes in their culture. The primary ingredient in the fermentation process of pulque was the Agave plant. As the early Spaniards ran out of brandy, they searched for a source of fermentable sugar for distilling. They experimented with the Agave, which was abundant in the volcanic soils in the Sierra Madre Region surrounding Guadalajara. The species that produced the most full-bodied taste was the Agave Tequilana, also known as the Blue Agave, or “Agave Azul” in Spanish.

There are 136 known species of Agave, but the only one used to produce tequila is Blue Agave. Fermentation products from the other species are not allowed to use the name “Tequila.” Norma Oficial Mexicana (NOM) in the 1970's. issued the official “tequila law”. It states that legal Tequila can only be produced in an area within roughly two hundred kilometers of Guadalajara The NOM also states that legal Tequila must be made from at least 51% Blue Agave in the fermentation mix.

Today, over 90,000 acres of Blue Agave are under cultivation in the Tequila-growing region of Mexico with the greatest concentration near the town of Tequila More than 52%, almost 82 million liters, of all Tequila is produced in the city of Tequila. The second-largest producing area is Arandas, with over 10% of production, followed by Guadalajara at 6.9%. The state of Tamaulipas makes about 43,000 liters and Guanajuato 177,000 liters. Today about 38,000 workers are employed in the industry, 33,000 of them farmers and field workers.

Beside the region of the Tequila State, the Aztecs Indians from the Oaxaca area, had cultivated a certain species of the agave plant for juice. They would ferment this into what is now called pulque. The juice was then pressed out, fermented for several days, and finally distilled. The result was named “mescal”. Technically, tequila is a type of mezcal, but mezcals are not tequilas. They both derive from varieties of the plant Agave and known to the natives as “Mexcalmetl” mezcal and pulque, both similar to Tequila but heavier in flavor, are distilled from sap taken from the roots, stalk, and leaves of wild Agave plants. Most mezcal is produced around the city of Oaxaca (and can officially be produced in the states of Guerrero, Durango, San Luis Potosi and Zacatecas).

Agave fields are planted from mecuates, small offshoots growing from the base of adult plants, but they can also be grown from seed. The offshoots are usually started in a nursery for a year and then transplanted to the fields. Usually 1,500-2,000 mecuates are planted to each acre and each plant requires 7-10 years to reach mature. When fully grown, the plant will reach a height of 5 to 6 feet. At this stage in the wild, Agave begins to grow the central flower-bearing stalk, which can grow to as much as 3 meters in height.

Flowers are pollinated naturally by long-nosed bats (Leptonycteris nivalis), and then the plant dies. Under cultivation, however, just before the stalk emerges, the field workers who harvest the Agave, remove the elongated, sharp pointed leaves with long-handled knives called coas, leaving the central core of the plant exposed. This core is called a pina and resembles a pinecone. The pina is allowed to continue growing, becoming riper and much larger. Harvested pinas can weigh from 50 to 150 pounds, and 500-pound pinas have been reported, but they are rare. The pinas are taken to the factories where traditional distillers split the pina in half with axes and stack them in ovens called hornos. There they are steamed for approximately 72 hours. After cooling another 24 hours, the pina is soft, fibrous and caramel-colored with a taste resembling that of honey-dipped yams. Larger distillers may shorten the steaming process by using pressure cookers. This process is much more rapid and takes only about 8 to 12 hours.

The pinas are then crushed by steel rollers or by a stone grinder, a large wheel of volcanic rock slowly drawn round and round by a mule or horse. Large distillers use a mechanical crusher, which resembles a wood chipper. The pinas are then minced and strained to remove the remaining sugar. This extricated juice constitutes the basis of all Tequila, and the left over extracted Agave have been treated as waste material for as long as the first Tequila ever been produced.

More than 100 million of these Agave plants cover the hills of the Sierra Madre region, west of Guadalajara. The Agave plants are lined up in rows roughly 1,000 to an acre. The total weight from the after extracted Agave pinas is estimated for more than 200,000 ton per year. Except some local furniture manufacturers using them as mattress cousin or filler material, there are very little commercial or industrial application toward the Agave waste material at this moment; more than 98% of the waste material are dumped into land fill or burned as fuel.

Blue Agave is the most important crop in western Mexico due to its major role in the Tequila industry, which provides thousands of Mexicans with a livelihood with a history that can be traced for over 300 years and allows the workers to proudly display a product that is purely and truly Mexican.

Of the more than 50 Tequila factories in the state of Jalisco most are minor operations. About ten companies, largely those in the town of Tequila account for three-fourths of total production. Sales in the United States have increased over 1500% between 1975 and 1995, making the U.S. the world's largest importer, accounting for 90% of all Tequila exports. Canada, France and Japan are the next largest importers.

Polyurethane

Polyurethane is a polymer consisting of the repeating unit —[—ROOCNHR′—]—ₙ [8R8OOCNH8R′8]ₙ, where R may represent a different alkyl group than R′. Alkyl groups are chemical groups obtained by removing a hydrogen atom from an alkaline, a hydrocarbon containing all carbon-carbon single bonds. Most types of polyurethane resin cross-link and become thermosetting plastics. However, some polyurethane resins have a linear molecular arrangement that does not cross-link, resulting in thermoplastics.

Thermosetting polyurethane molecules cross-link into a single giant molecule. Thermosetting polyurethane is widely used in various forms, including soft and hard foams. Soft, open-celled polyurethane mixers foams are used to make seat cushions, mattresses, and packaging. Hard polyurethane mixers are used as insulation in refrigerators, freezers, and homes furnishing goods or appliances bodies. Thermoplastic polyurethane molecules have linear, highly crystalline molecular structures that form an abrasion-resistant material. Thermoplastic polyurethane mixers are molded into shoe soles, car fenders, door panels, and other products.

Phenolic

Phenolic (phenol-formaldehyde) resins, first commercially available in 1910, were some of the first polymers made. Today, Phenolics are some of the most widely produced thermosetting plastics. They are produced by reacting phenol (C₆H₅OH) with formaldehyde (HCOH). Phenolics plastics are hard, strong, inexpensive to produce, and they possess excellent electrical resistance. Phenolics resins cure (cross-link) when heat and pressure are applied during the molding process. Phenolics resin-impregnated paper or cloth can be laminated into numerous products, such as electrical circuit boards. Phenolics resins are also compression molded into electrical switches, pan and iron handles, radio and television casings, and toaster knobs and bases.

Melamine-Formaldehyde and Urea-Formaldehyde

Urea-formaldehyde (UF) and melamine-formaldehyde (MF) resins are composed of molecules that cross-link into clear, hard plastics. Properties of UF and MF resins are similar to the properties of Phenolics resins. As their names imply, these resins are formed by condensation reactions between urea (H₂NCONH₂) (H2NCONH2) or melamine (C3H6N6) (C3H6N6) and formaldehyde (CH₂O) (CH2O)

Melamine-formaldehyde resins are easily molded in compression and special injection molding machines. MF plastics are more heat-resistant, scratch-proof, and stain-resistant than urea-formaldehyde plastics are. MF resins are used to manufacture dishware, electrical components, laminated furniture veneers, and to bond wood layers into plywood.

Urea-formaldehyde resins form products such as appliance knobs, knife handles, and plates. UF resins are used to give drip-dry properties to wash-and-wear clothes as well as to bond wood chips and wood sheets into chip board and plywood.

Amino Resin

Amino resins are versatile, low-cost cross-linkers for today's high-performance thermosetting coatings. A wide variety of amino products are available that offers advantage of flexibility in supply and price competitiveness in selecting the most suitable product for an application. Choosing the best cross-linker requires knowledge of its structure, including functional groups available to participate in reactions and molecular weight which is similar or adaptable to the fiber of blue agave residue. Amino resins react readily with primary and secondary hydroxyl, carboxyl and amide-functional polymers, it creates strong and stable three dimensional bonding among the fiber of Blue Agave and other adding mixers. This is accomplished through reacting the amino resin with functional groups on the film-former and simultaneous self-condensation with other amino molecules.

Starch

Granular water-insoluble starch is added as an inert filler to complete the process. Starch is used primarily as a binding material to glue or adhere solid constituents together in forming a hetero-genius mixture of components. The polarized character of the fiber of Blue Agave residue is chemically compatible with starch, which is highly polarized due to existence of hydroxyl groups on approximately half of carbon atoms. Having fiber of Blue Agave residue represent as largest active component in the blend, Either an animal grease, glycerin or water are necessary to be blended in the process as a catalyst. Typically, plasticizers used to enable the formation of starch melts are either highly volatile liquids at the melting point such as water, or low volatile liquids such as glycerin. In the process of formulating Tequilastic, the animal greace or glycerin or water will react with the granular starch mixer to form a new chemical bonding. Starch is used as a binder, as a thermo plastically processible constituent within the fiber of Blue Agave residue and other thermoplastic polymer blends, and as a thermoplastic material in itself.

Examples of patents that demonstrates the use of starch as a binder and, in particular, processes for molding articles from aqueous starch mixtures include U.S. Pat. No. 5,660,900 to Andersen et al.; U.S. Pat. No. 5,683,772 to Andersen et AL.; U.S. Pat. No. 5,709,827 to Andersen et al. ; U.S. Pat. No. 5,868,824; and U.S. Pat. No. 5,376,320 to Tiefenbacher et al. For purposes of disclosing compositions, methods, and systems for molding aqueous starch mixtures that are subsequently dried so as to form a binding matrix of dried starch which binds together concrete solid materials such as fibers and particulate fillers. The foregoing patents have been incorporated herein for specific reference.

There are circumstances where attempts were taking place to use starch as a thermoplastic material, either alone or as a component within thermoplastic blends. Native starch does not typically behave as a thermoplastic material in itself but has to be heated in presence of plasticizers. Typically, the plasticizer has to be a liquid (at least when raised to the resulting gelatinization or melting point of starch) and it also has to be somewhat polarized in order to be chemically compatible with starch, which is itself highly polarized due to the existence of hydroxyl groups on approximately half of carbon atoms. Typically, plasticizers used to enable the formation of starch melts are either highly volatile liquids at the melting point such as water, or low volatile liquids such as glycerin.

Starch melts using water as the plasticizing solvent have been referred in art as “destructurized starch”. Starch is said to be “destructurized” because it ceases to be a solid granular particulate as found in its native state. Moreover, it is said to be “destructurized” because the dissolution or melting of starch in the presence of water is an irreversible process. Starch that has been dissolved into or melted in the presence of water can never return to its native, granular state. Upon re-solidification of a melt of destructurized starch, typically by cooling below its melting or softening point, it will yield an essentially amorphous or semi-crystalline starch material that is self-supporting or “form stable”, but only so long as water content is kept above at least 5% by weight of the starch and water mixture during the entire process including during cooling, preferably above at least 10%. Otherwise, the starch will tend to re-crystallize into a brittle material instead of forming a more amorphous and less brittle solid.

The use of “destructurized starch” as a commercial thermoplastic material has been limited for a number of reasons, including difficulty in processing, poor long term mechanical properties, high sensitivity to fluctuations in ambient moisture, including poor dimensional stability, and the difficulty of forming homogeneous blends of destructurized starch with more hydrophobic polymers that are less sensitive to fluctuations in moisture. Examples of patents that disclose the manufacture of “destructurized starch” and blends of destructurized starch and other polymers include U.S. Pat. No. 4,673,438 to Wittwer et AL.; U.S. Pat. No. 4,900,361 to SACHETTO et al. ; U.S. Pat. No. 5,095,054 to Lay ET AL.; U.S. Pat. No. 5,256,711 TO TOKIWA ET AL.; U.S. Pat. No. 5,275,774 to Bahr et AL.; U.S. Pat. No. 5,382,611 to Stepto et AL; U.S. Pat. No. 5,405,564 to Stepto et AL.; and U.S. Pat. No. 5,427,614 to Wittwer et al. For purposes of disclosing compositions and methods for manufacturing “destructurized starch” compositions, including blends of “destructurized starch” and other polymers, the foregoing patents are incorporated herein by specific reference.

Others have shown that it is preferable to greatly reduce the amount of water in starch melts by replacing the water inherently found in starch with an appropriate low volatile plasticizers capable of causing starch to form a thermoplastic melt below its decomposition temperature, such as glycerin, polyalkylene oxides, mono-and diacetates of glycerin, sorbitol, and citrates. In the process of formulating Tequilastic, we have discovered that natural fiber from Blue Agave residue has a very strong tensile strength, the newly formed Tequilastic is able to resist abrasive and impact from outside at a much higher level.

Natural fiber from Blue Agave allows for improved processability, greater mechanical strength, better dimensional stability over time, and greater ease in blending the starch melt with other polymers compared to “destucturized starch”. Thermoplastic starch material in which most or all of water is replaced by a low volatile plasticizers, either before or during the process, are variously referred to as “thermo plastically processible starch” and “thermoplastic starch”.

Every year in the United States alone, individuals trash millions of tons of plastics. Of the estimated 190 million metric tons (420 billion pounds) of municipal waste collected annually in the United States, about 9 percent are plastics. Landfill after landfill reach their full capacity, additional landfill spaces are taken with new available landfill spaces diminished across the United States. Accordingly, alternative methods for reducing and disposing of wastes properly, including plastics, have been aggressively explored. Some of these options include reducing consumption of plastics, use of biodegradable plastics, and incinerating or recycling of plastic waste.

Biodegradable Plastics

Most plastics cannot readily break down into simpler components due to their molecular stability. Plastics are therefore generally not considered biodegradable. However, biodegradable plastics such as Tequilastic will disintegrate over specified period of time due to bacterial actions or exposure to sunlight. For example, by incorporating starch molecules into certain thermosetting plastic resins in a manufacturing process will allow commonly occurring bacteria to “eat” into starch molecules when same plastics are collected in landfills. This process causes polymer molecules to break apart, allowing Tequilastic to decompose. Exposure to sunlight is another method that Tequilastic can disintegrate. Prolonged exposure to ultra-violet radiation from the sun makes Tequilastic molecules to become brittle and slowly break apart. We are creating specific Tequilastic that will degrade faster with exposure to sunlight, but slow down the biodegradable process during its life cycle of normal use.

Incineration

Certain solid wastes such as paper, plastics, wood, and other flammable materials can be burned in incinerators. The resulting ash demands much less space for disposal than the original waste in the solid form would otherwise require. But incineration of plastics is capable of producing hazardous emissions and other pollutants into the air. The incinerations method has been regulated and discouraged where possible. The present invention relates to compositions and methods for manufacturing Tequila (Blue Agave) residue formulated compositions and articles made by the Tequila (Blue Agave) formulated compositions. The idea and concept of applying and re-deploy a natural waste material to produce other forms of biodegradable articles is aimed to generate environmental benefits and economical value to the mankind. The unique characters of Tequila (Blue Agave) residue together with its distinct fiber strength offer a variety of commercial applications.

Articles produced by biodegradable thermosetting Tequila (Blue Agave) residue formulated composition can start to relieve high demand for petrochemical plastic consumption and capable to reduce environmental pollution associated with plastic waste. With an upward of more than two hundred thousand (200,000) tons of Tequila (Blue Agave) residue generated and become available per year for collection and recycle, Tequilastic production is feasible for both mass production and commercialization. It is a low cost, affordable and environmental friendly substitute for certain plastic products.

The present invention relates to a biodegradable thermosetting resin composition comprising a carbohydrate biomass substance supplied by coffee bean waste or from a blend of Tequila (Blue Agave) residue with starch.

The present invention relates to a biodegradable thermosetting resin composition comprising a carbohydrate biomass substance from Tequila (Blue Agave) residue. The waste material is washed with water and dried to achieve less than 15% moisture content. The resulting composition is ground into powder having a fine particle size of between 40 um to 100 um in meeting criteria for different thermosetting applications. The present invention relates to some thermosetting resins which are polyurethane, phenol, melamine formaldehyde, urea formaldehyde or amino.

The present invention relates to a biodegradable thermosetting resin composition comprising a carbohydrate biomass substance and at less one or more blending reactive substance having the melting point of between 90 to 130 degree C.

The biodegradable thermosetting resin composition contains a carbohydrate biomass substance blends with melamine, phenol, urea, amino or polyurethane and which reactive substance having the melting point range between of 90 to 130 degree C. in the presence of a catalyst.

The biodegradable thermosetting resin composition contains a carbohydrate biomass substance that blends with melamine, phenol, amino or polyurethane and which reactive substance having the melting point range between of 90 to 130 degree C. in the presence of a catalyst and the catalyst is either an animal grease, glycerin or water.

### EXAMPLE 1

### A Method for Making Biodegradable Tequilastic Kitchenware

In this process, we used Tequila Blue Agave residue as the major component in the composition. The Tequila Blue Agave residue was rinsed in clean tap water, and then dried and ground to a particle size of 40 to 100 microns. The ground Tequila Blue Agave residue was then thoroughly mixed with starch and amino resin, to produce a substantially homogeneous blend. The formula is based on the concentration range for the Tequila Blue Agave fiber powder of approximately 30% to 60% by weight of solid, for the starch of approximately 5% to 10% by weight of solids, for the melamine-formaldehyde resin of a range between 10% and 40% by weight of solids. We added glycerin in the blending by weight of 3% to 7% as a catalyst. All materials blended in the composition were similar in granule size in order for achieve the most even mix in the blending process; and the moisture content was kept between 5% and 8%. A mould was pre-heated for at least 20 minutes before starting the thermosetting process. Mold pressure was kept between 10 psi and 120 psi. Temperature was kept between 90 degrees and 130 degrees C. The thermosetting time required was not less than two minutes, and not more than eight minutes. Cooling time is required after the Tequilastic article is formed, and the product is preferably polished to provide a final smooth finish.

### EXAMPLE 2

A method to make a thermosetting biodegradable Tequilastic building and home construction/renovation material. In this process, we used as the major component in the composition. The Tequila Blue Agave residue was rinsed with clean tap water and then ground to a particle size between 40 and 100 microns. The ground Tequila Blue Agave residue powder was then mixed with starch, melamine-formaldehyde resin, talc, calcium carbonate and glycerin to yield a substantially homogeneous blend. The formula included a concentration range for the Tequila Blue Agave residue of approximately 20% to 60% by weight, for the starch of approximately 0% to 5% by weight, for the melamine resin of a range between 5% and 45% by weight, for calcium carbonate of between 5% and 15% by weight, for talc of a range between 1% and 5% by weight of solid, and for glycerin a concentration range between 3% and 7%. All the ingredients were provided in a particle size similar to the fiber of Tequila Blue Agave residue, in order to achieve the most even mix in the blending process. The moisture content of the mixture was kept between 5% and 15%. The mold was pre-heated for 20 minutes before starting the thermosetting process. Pressure in the mold was kept between 50 psi and 250 psi; and the temperature was kept between 90 degrees C. and 130 degrees C. The thermosetting time required was not less than 3 minutes, and not more than 8 minutes. Cooling time was required after the Tequilastic building and construction/renovation materials were formed. Both rough and fine polishing are preferable, to deliver a smooth, glossy finish.

### EXAMPLE 3

### A Method to Make a Thermosetting Biodegradable Tequilastic Interior Floor Plank Material

The Tequila Blue Agave residue was rinsed with clean tap water and then ground to a particle size between 40 and 100 microns. The ground Tequila Blue Agave residue powder was then mixed with melamine-formaldehyde resin, calcium carbonate and glycerin to yield a substantially homogeneous blend. The formula included a concentration range for the Tequila Blue Agave residue of approximately 20% and 60% by weight, for the melamine-formaldehyde resin of a range between 25% and 45% by weight, for calcium carbonate of between 5% and 15% by weight, and for glycerin a concentration range between 3% and 7%. All the ingredients were provided in a particle size similar to the fiber of Tequila Blue Agave residue, in order to achieve the most even mix in the blending process. The moisture content of the mixture was kept between 5% and 8%. The mold was pre-heated for 20 minutes before starting the thermosetting process. Pressure in the mold was kept between 50 psi and 250 psi; and the temperature was kept between 90 degrees C. and 130 degrees C. The thermosetting time required was not less than 3 minutes, and not more than 8 minutes. Cooling time was required after the Tequilastic floor plank materials were formed. Both rough and fine polishing are preferable, to deliver a smooth, glossy finish.

We claim:

1. A composition comprising agave residue and a thermosetting polymer.

2. An article of manufacture comprising a composition as in claim 1.

3. A kitchenware article comprising a composition as in claim 1.

4. A building material comprising a composition as in claim 1.

5. A decorative article comprising a composition as in claim 1.

6. A packaging material comprising a composition as in claim 1.

7. A method comprising the steps of
mixing powdered agave residue with a thermosetting polymer; and
curing the mixture in a mold to form an article of manufacture.

8. A method as in claim 7 wherein said powder has a particle size of 40 to 100 microns.

9. A method as in claim 8 wherein said curing step includes a temperature between 90 and 130 degrees C. and a pressure between 10 psi and 120 psi.

10. A method comprising the steps of:
mixing blue agave residue and starch with a thermosetting resin, said residue having a particle size of 40 to 100 microns;
and curing the mixture in a mold for two to seven minutes, at a temperature between 90 and 130 degrees C. and a pressure of 10 psi to 120 psi.

11. A method as in claim 10, further including the step of applying a second layer of thermosetting composition to the product produced, and returning the product and second layer to the mold for a second cure.
